(19) United States
(12) Patent Application Publication
(10) Pub. No.: US 2018/0153572 A1
(43) Pub. Date: Jun. 7, 2018

(54) APPARATUS AND METHODS FOR RETRIEVING OBJECTS FROM WITHIN THE BODY OF A SUBJECT

(71) Applicant: Transit Scientific, LLC, Salt Lake City, UT (US)

(72) Inventors: Shawn P. Fojtik, Park City, UT (US); Stefan Niederauer, Salt Lake City, UT (US); Hallie Thorp, Salt Lake City, UT (US)

(21) Appl. No.: 15/614,474

(22) Filed: Jun. 5, 2017

Related U.S. Application Data
(60) Provisional application No. 62/317,614, filed on Apr. 3, 2016.

(51) Int. Cl. A61B 17/3205; A61B 17/221; A61B 8/08; A61B 17/50

(52) U.S. Cl. CPC A61B 17/32056; A61B 17/221; A61B 2017/00358; A61B 17/50; A61B 8/0833

(57) ABSTRACT

A medical device for removing or retrieving objects from the body of a subject (e.g., from a vessel and/or a cavity in the body of the subject. Such a retrieval apparatus includes a handle, a sheath protruding from the handle, and a snare. The snare includes a loop that may extend from a distal end of the sheath, as well as an intermediate portion that resides within a lumen of the snare and one or more ends that are coupled to or are otherwise associated with one or more controls carried by the handle. The controls may enable retraction of the loop into the lumen of the sheath, sizing of the loop, and reciprocation, or back and forth movement, of the snare and its loop. Methods for using such a retrieval apparatus are also disclosed.

### CROSS-REFERENCE TO RELATED APPLICATION

A claim for priority to the Apr. 3, 2016 filing date of U.S. Provisional Patent Application No. 62/317,614, titled SURGICAL SNARE (“the '614 Provisional Application”) is hereby made pursuant to 35 U.S.C. § 119(e). The entire disclosure of the '614 Provisional Application is hereby incorporated by reference.

### TECHNICAL FIELD

This disclosure relates generally to medical devices for retrieving and removing objects from a vessel and/or cavity in the body of a subject. More specifically, this disclosure relates to retrieval apparatuses that include sheaths and snares with loops that may be sized and/or retracted toward or into the sheaths. This disclosure also relates to retrieval apparatuses with snares that may reciprocate, or move back and forth. For the sake of simplicity, a retrieval apparatus according to this disclosure may be referred to as a “snare.”

### RELATED ART

Occasionally, a foreign object may be present within a vessel or a cavity of a subject's body. Sometimes, the foreign object may be naturally formed (e.g., an embolism, a stone, etc.). Other times, the foreign object may be totally foreign to the subject's body. An example of this type of foreign object includes, but is not limited to, a piece that has broken off of a medical device (e.g., a guide wire, a catheter, etc.) and remains within the subject's body after the remainder of the medical device has been removed from the subject's body. Alternatively, a foreign object may comprise a medical device that has been temporarily placed within a subject's body, which is intended to be subsequently removed from the subject's body.

There are a variety of reasons for removing different types of foreign objects from a subject's body. To provide a few examples, foreign objects may be removed to reduce or eliminate pain, to improve flow, to prevent septicemia, to prevent perforation or other physical damage to the inside of a subject's body, as well as for a number of other reasons.

A variety of apparatuses have been developed to remove or retrieve foreign objects from the body of a subject (e.g., a human, animal, etc.). For the sake of simplicity, such an apparatus is also referred to herein as a “retrieval apparatus.” Typically, a retrieval apparatus includes a snare and catheter. A snare usually includes an elongate element with a loop at its distal end. As indicated, a snare is typically used in conjunction with a catheter, which enables the snare to be introduced to a particular location within a subject's body; for example, a location where a foreign object may reside. With a catheter properly positioned within the subject's body (i.e., when a tip of the catheter has been positioned in the general area of a foreign object to be removed from the subject's body), the elongate element of the snare may be advanced, or pushed, distally through the catheter to extend the loop distally from a distal tip of the catheter. The loop may then be drawn proximally in an effort to engage the foreign object. Repeated distal and proximal movements of the loop are often required, as the process of engaging a foreign object is often performed blindly and does not provide an operator (e.g., a healthcare professional, etc.) with much, if any, control. Thus, the process of engaging a foreign object within the body of a subject is one of trial and error. Once the foreign object has been engaged, it may be captured between the loop and the distal end of the catheter, and the catheter and the snare may be removed from the subject's body, hopefully bringing the foreign object along with them.

Conventionally, capturing a foreign object between a loop and the distal end of a catheter and holding the foreign object in place requires that one person hold the catheter and the elongate element of the snare in place relative to one another, while another individual removes the assembly from the subject's body.

### SUMMARY

In various embodiments, an apparatus for removing or retrieving a foreign object from within a body of a subject, which is also referred to herein as a “retrieval apparatus” or, for the sake of simplicity, as a “snare,” includes a snare, a sheath, and handle for operating the snare.

The snare includes an elongated element, or shaft, and a capture element, such as a loop, at a distal end of the elongated element. One end of the snare, which may be referred to herein as a “first end” or as a “movable end” of the snare, may be associated with the handle, or, more specifically, with controls of the handle. The opposite end of the snare, which may be referred to as a “second end” of the snare, may be secured in place relative to another feature of the retrieval apparatus (e.g., a location along a length of the retrieval apparatus, such as a location within the handle, etc.) or it may be moveable (e.g., associated with a loop extension control, etc.). The loop of the snare is defined by a portion of a length of the elongated element of the snare and, thus, is located between the fixed end of the snare and the movable end of the snare.

The sheath is an elongated element with a distal end capable of being introduced into a body of a subject and placed at a desired location within the body of the subject, a proximal end that is intended to remain outside of the subject's body, and a lumen extending along at least a portion of a length of the sheath and opening at or adjacent to the distal end of the sheath. The lumen of the sheath is capable of receiving at least a portion of a length of the elongated element of the snare (e.g., a majority of the length of the elongated element, etc.). The lumen may also enable longitudinal movement of at least a portion of the length of the elongated element of the snare. A portion of the length of the snare that extends out of the distal end of the sheath may define the loop of the snare. The proximal end of the sheath may be coupled to the handle, with the movable end of the elongated element of the snare and a portion of the elongated element adjacent to the movable end extending into the handle.

The handle, which receives at least the first end of the elongated element of the snare and optionally receives the second end of the elongated element of the snare, may include one or more controls. As an example, the handle may include a loop extension control. The loop extension control may enable advancement of the loop out of the lumen of the sheath, causing the loop to extend from the distal end of the sheath. The loop extension control may also enable retraction of the loop into the lumen of the sheath. The loop extension control may, therefore, enable sizing of the loop.

As another example, the handle may include a loop size control to which the movable end of the elongated element of the snare is secured. Movement of the loop size control may determine a size of the loop of the snare (e.g., a length of a portion of the elongated element of the snare that extends from the distal end of the sheath, etc.). The loop size control may be capable of locking the movable end of the elongated element of the snare into a position that will maintain or substantially maintain (e.g., allowing for small movements in the elongated element of the snare and, thus, in the portion of the elongated element that forms the loop, etc.) the size of the loop. In some embodiments, a loop size control may also be capable of advancing or extending the loop distally out of the lumen of the sheath and of proximally retracting the loop at least partially into the lumen of the sheath.

In some embodiments, the handle may include a reciprocating control. The reciprocating control may be associated with the elongated element of the snare in a manner that enables back-and-forth, or reciprocating or oscillating, movement of the elongated element. Reciprocating movement in, or reciprocation of, the elongated element may include reciprocation of the portion of the length of the elongated element that defines the loop. Reciprocation of the portion of the length of the elongated element that defines the loop may facilitate movement of the loop to a desired location within a body of a subject, movement of the loop over and/or around a targeted object within the body of the subject, and/or use of the loop to cut through tissue or a foreign body within the body of the subject.

Methods for using a retrieval apparatus, or snare, are also disclosed. In various embodiments, such a method may include introducing a distal end of a sheath of the snare, as well as the loop, into the body of a subject. The distal end of the snare may be advanced (distally) into the subject's body to a desired location, or a target location; for example, a location from which an object or tissue is to be removed. The loop may then be moved onto and engage the object that is to be removed from the body, and the snare and the object may then be pulled from the body.

In embodiments where the loop can be retracted into the distal end of the sheath, the loop may be retracted into the distal end of the sheath before the snare is introduced into the body of a subject. Once the distal end of the snare is in place at the desired location within the body, the loop may be advanced distally out of the distal end of the sheath, and then moved in place over the object that is to be removed from the body. Once the loop is in place around the object, the loop may be at least partially retracted into the distal end of the sheath, which may enable the loop (and the sheath) to further engage the object and, thus, to secure the object in place as the snare is removed from the body.

In embodiments where a size of the loop can be adjusted, the size of the loop may be set prior to introduction of the distal end of the snare into the subject's body. Such sizing of the loop may be based on the size of the object, or on the size of a feature of the object, that is to be removed from the subject's body. Alternatively, the loop may be sized or re-sized once the distal end of the snare has been positioned at the desired, or target, location with the subject's body. Sizing or re-sizing of the loop may occur with the aid of devices that enable the desired location and the object to be visualized (e.g., under fluoroscopy, under ultrasound, etc.). Sizing or re-sizing of the loop within the subject's body may occur before the loop is placed around an object that is to be removed from the body or after the loop has been placed around the object.

A method of using the snare may include reciprocation or oscillation of an elongated element of the snare and, thus, of the loop. The loop may be reciprocated to facilitate introduction of the distal end of the snare (and the loop) to a desired, or target, location within the body of the subject, to facilitate placement of the loop over an object that is to be removed from the body, to facilitate engagement of an object by the loop, or to cut into and/or through an object over which the loop has been placed.

Features and advantages of various aspects of the disclosed subject matter, as well as other aspects of the disclosed subject matter, will become apparent to those of ordinary skill in the art through consideration of the ensuing description, the accompanying drawings, and the appended claims.

### BRIEF DESCRIPTION OF THE DRAWINGS

In the drawings:

FIGS. 1 and 1A provide a representation of an embodiment of a retrieval apparatus, or snare, according to this disclosure;

FIG. 2 shows an example of an initial position of a loop control button on the exterior of the handle of an embodiment of retrieval apparatus;

FIGS. 2A and 2B are cross-sectional representations depicting various components of the embodiment of retrieval apparatus shown in FIG. 2, with FIG. 2A providing a representation taken along a length of the embodiment of retrieval apparatus of FIG. 2 and FIG. 2B providing a representation taken along the width of the embodiment of retrieval apparatus of FIG. 2;

FIGS. 3-3B show an example of use of the loop control button on the exterior of the handle of the embodiment of retrieval apparatus illustrated by FIG. 2 to size a loop of the retrieval apparatus;

FIG. 4 shows an example of a sized position of the loop control button on the exterior of the handle of the embodiment of retrieval apparatus illustrated by FIG. 2;

FIGS. 5-6B show another embodiment of retrieval apparatus and its components and features, with FIG. 5 providing perspective view of the retrieval apparatus, FIG. 6 providing an assembly view of components carried by a housing of the embodiment of retrieval apparatus shown in FIG. 5, FIG. 6A providing an exploded view of the components carried by the housing of the embodiment of retrieval apparatus shown in FIG. 5, and FIG. 6B providing a perspective view of a shuttle that enables the selection of a size of a loop of a snare of the embodiment of retrieval apparatus shown in FIG. 5; and

FIG. 7 schematically depicts and embodiment of use of a retrieval apparatus, or snare, to remove an object from a body of a subject.

### DETAILED DESCRIPTION

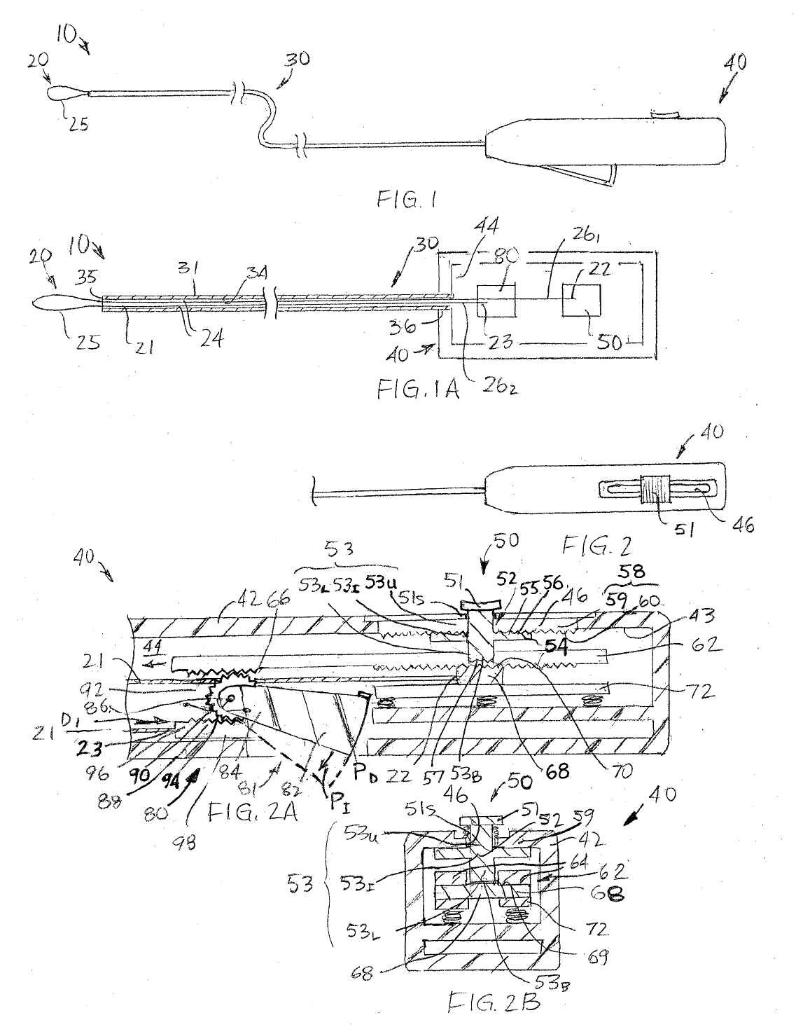

As illustrated by FIGS. 1 and 1A, a retrieval apparatus 10 according to this disclosure may include a snare 20, a sheath 30, and a handle 40. A majority of the snare 20 may be carried by the sheath 30. The sheath 30 may be coupled to the handle 40. The handle 40 may be configured to enable manipulation of the sheath 30 as the sheath 30 and the snare 20 are introduced into a subject's body and advanced to a desired location, or a target location, with the subject's body. In addition, the handle 40 may include controls 50, 80, etc., that enable an individual (e.g., a healthcare provider, such as a physician, a physician's assistant, a nurse, etc.) to manipulate the snare 20 in a desired manner. A configuration of the handle 40 may enable an individual to introduce and advance the sheath 30 and operate the snare 20 with one hand.

With continued reference to FIG. 1A, the snare 20 of the retrieval apparatus 10 may comprise an elongated element 21, such as a line, a wire, or a cable, which may be formed from any suitable material (e.g., a polymer, a metal or metal alloy, etc.). The elongated element 21 includes a first end 22, a second end 23 opposite from the first end 22, and an intermediate portion 24 between the first end 22 and the second end 23. A portion of a length of the intermediate portion 24 of the elongated element 21 may define a loop 25.

The sheath 30 of the retrieval apparatus 10 may comprise a tubular element, such as a catheter. As such, the sheath 30 may include an outer wall 31 that defines one or more lumens 34. Each lumen 34 may extend along an entire length of the sheath 30, or along at least a distal portion of the sheath 30. In either of these embodiments, the lumen 34 may open to a distal end 35 of the sheath 30, with the distal end 35 being the end of the sheath 30 that is introduced into a subject's body and advanced to a desired location within the subject's body. The opposite end of the sheath 30 is its proximal end 36. The proximal end 36 of the sheath 30 may be coupled to the handle 40 of the retrieval apparatus 10. In embodiments where the lumen(s) 34 extend(s) through the sheath 30 along the entirety of its length, the lumen(s) 34 may open to and, thus, communicate with an interior of the handle 40.

As depicted by FIG. 1A, a majority of the length of the intermediate portion 24 of the elongated element 21 of the snare 20 resides within the lumen(s) 34 of the sheath 30. A section of the intermediate portion 24 that defines the loop 25 may be located outside of the lumen(s) 34 and, thus, extend or protrude from the distal end 35 of the sheath 30. In some embodiments, the loop 25 may be defined as a section of the intermediate portion 24 of the elongated element 21 of the snare 20 is advanced distally out of the lumen(s) 34 of the sheath 30. The first end 22 of the elongated element 21 of the snare 20 may extend proximally out of a lumen 34 at the proximal end 36 of the sheath 30 and into an interior 44 of the handle 40, where the first end 22 of the elongated element 21 and, optionally, an adjacent, proximal portion 26₁ of the elongated element 21 may be associated with one or more controls 50, 80 of the handle 40. In some embodiments, the second end 23 of the elongated element 21 of the snare 20 may also extend out of a lumen 34 at the proximal end 36 of the sheath 30 and into the interior 44 of the handle 40, where the second end 23 of the elongated element 21 may be secured to a feature of the handle and, optionally, an adjacent, proximal portion 262 of the elongated element 21 may be associated with one or more of the controls 50, 80 of the handle 40.

Embodiments of the manner in which controls 50, 80 of the handle 40 of a retrieval apparatus 10 may interact with the snare 20 are described in reference to FIGS. 2-4.

FIG. 2 shows a button 51 of an embodiment of a loop size control 50 of a handle 40 of a retrieval apparatus 10 according to this disclosure, with the button 51 in an “initial position” relative to an elongated slot 46 formed through a portion of a body 42, or a housing, of the handle 40. The initial position of the button 51 depicted by FIG. 2 is merely illustrative, as the button 51 may be located at any position along the length of the slot 46 in its initial position. In its initial position along the length of the elongated slot 46, however, the button 51 is in a default orientation, or an initial state, as illustrated by FIGS. 2A and 2B. A spring 51_{S} or another suitable element may bias, or force, the button 51 to its default orientation.

FIGS. 2A and 2B also depict other features of the loop size control 50 while the button 51 is in its default position. Specifically, FIG. 2A shows the loop size control 50 as including an actuator 52 on which the button 51 is located, a button position retainer 58 capable of selectively interacting with the actuator 52, a runner 62, and a shuttle 66 capable of selectively interacting with the runner 62. As shown, the first end 22 of the elongated element 21 of the snare 20 (FIG. 1A) is coupled to the shuttle 66. A location of the shuttle 66 along a length of the runner 62 determines a position of the first end 22 of the elongated element 21 of the snare 20. Thus, the position of the shuttle 66 along the length of the runner 62 may also define a distance the loop 25 (FIGS. 1 and 1A) of the snare 20 extends beyond the distal end 35 (FIGS. 1 and 1A) of the sheath 30 and/or the size of the loop 25.

The actuator 52 protrudes (e.g., downwardly, in the orientation depicted by FIGS. 2A and 2B) from the button 51. More specifically, a shaft 53 of the actuator 52 protrudes from the button 51. A first portion 53_{U} of the shaft 53 (e.g., an upper portion of the shaft 53, in the orientation depicted by FIGS. 2A and 2B) may be received within the elongated slot 46 that is defined through and extends along the body 42 of the handle 40, and between a pair of retention strips 59 of the button position retainer 58. The retention strips 59 may comprise parts of an interior surface 43 of the body 42 of the handle 40 or they may be secured to the interior surface 43 of the body 42. The retention strips 59 may be oriented parallel to one another on opposite sides of the elongated slot 46, adjacent to opposed edges of the elongated slot 46. The first portion 53_{U} of the shaft 53 of the actuator 52 may have a shape that, along with the opposed edges of the elongated slot 46 and/or opposed edges of the retention strips 59, prevents the shaft 53 and, thus, the actuator 52 from rotating.

The button position retainer 58 may also include a series of teeth 60 may protrude from each retention strip 59 (e.g., downwardly, in the orientation depicted by FIGS. 2A and 2B) along its length. The teeth 60 of the retention strips 59 may be capable of engaging complementary series of teeth 56 that protrude from surfaces 55 (e.g., upper surfaces, in the orientation depicted by FIGS. 2A and 2B) of wings 54 that extend laterally from opposite sides of an intermediate, second portion 531 of the shaft 53.

A third portion 53L of the shaft 53 (e.g., a lower portion of the shaft 53, in the orientation depicted by FIGS. 2A and 2B) may extend through a slot 63 that extends centrally through the length of the runner 62. Teeth 57 may protrude (e.g., downwardly, in the orientation depicted by FIGS. 2A and 2B) from an end 53_{B} of the shaft 53 (e.g., the bottom end of the shaft 53, in the orientation depicted by FIGS. 2A and 2B).

The runner 62 may be aligned with, but spaced apart from, the button position retainer 58. In some embodiments, a position of the runner 62 may be fixed relative to the body 42 of the handle 40. In other embodiments, including embodiments where reciprocation or oscillation of the snare 20 (FIG. 1A) is possible, the runner 62 may also be capable of such movement, i.e., of back-and-forth movement along its length. Movement of the runner 62 along its length may be enabled in any suitable manner, including, without limitation, by way of elongated recesses (not shown) in a portion of the body 42 of the handle 40 that receive opposite edges (not shown) of the runner 62.

The runner 62 may include a pair of elements 64 that are spaced apart from one another and oriented parallel to each other. The spaced apart, opposed edges of the elements 64 of the runner 62 may define the slot 63 through the runner 62. Each element 64 of the runner 62 may include teeth 66 that protrude (e.g., downwardly, in the orientation depicted by FIGS. 2A and 2B) from a surface 65 that faces away from the button position retainer 58.

A shuttle 68 may be located adjacent to surfaces 65 of the elements 64 of the runner 62, with a surface 69 of the shuttle 68 (e.g., an upper surface of the shuttle 68, in the orientation depicted by FIGS. 2A and 2B) facing the surfaces 65 of the elements 64 of the runner 62. A series of teeth 70 may protrude from the surface 69 of the shuttle 68, and may be capable of engaging complementary teeth 66 of the runner 62, as well as complementary teeth 57 protruding from the end 53_{B} of the shaft 53 of the actuator 52.

The shuttle 68 may be capable of travelling along a length of the runner 62. In some embodiments, the shuttle 68 may be capable of movement a track 72 or a similar element aligned with the runner 62.

The shuttle 68 may be biased, or forced, toward the runner 62 (e.g., upwardly in the orientation depicted by FIGS. 2A and 2B). In embodiments where movement of the shuttle 68 is confined by a track 72 or a similar element, that element may be biased, or forced, toward the runner 62 (e.g., by way of one or more springs, etc.), thereby forcing the shuttle 68 toward the runner 62.

As illustrated by FIGS. 2A and 2B, when the button 51 is in its default position (e.g., due to force applied by a spring 51_{S} associated with the button 51, etc.), the actuator 52 is also in a default orientation. In its default orientation, the actuator 52 is forced against the button position retainer 58, with teeth 56 of the actuator 52 engaging teeth 60 of the button position retainer 58, thereby holding the button in place along the length of the elongated slot 46 in the body 42 of the handle 40. When the actuator 52 is in the default orientation, the end 53_{B} of the shaft 53 of the actuator 52 is spaced apart from the surface 69 of the shuttle 68, which enables force on the shuttle 68 to push it toward the runner 62, and teeth 70 of the shuttle 68 to engage teeth 66 of the runner 62, thereby enabling the runner 62 to secure the shuttle 68 in position along the length of the runner 62.

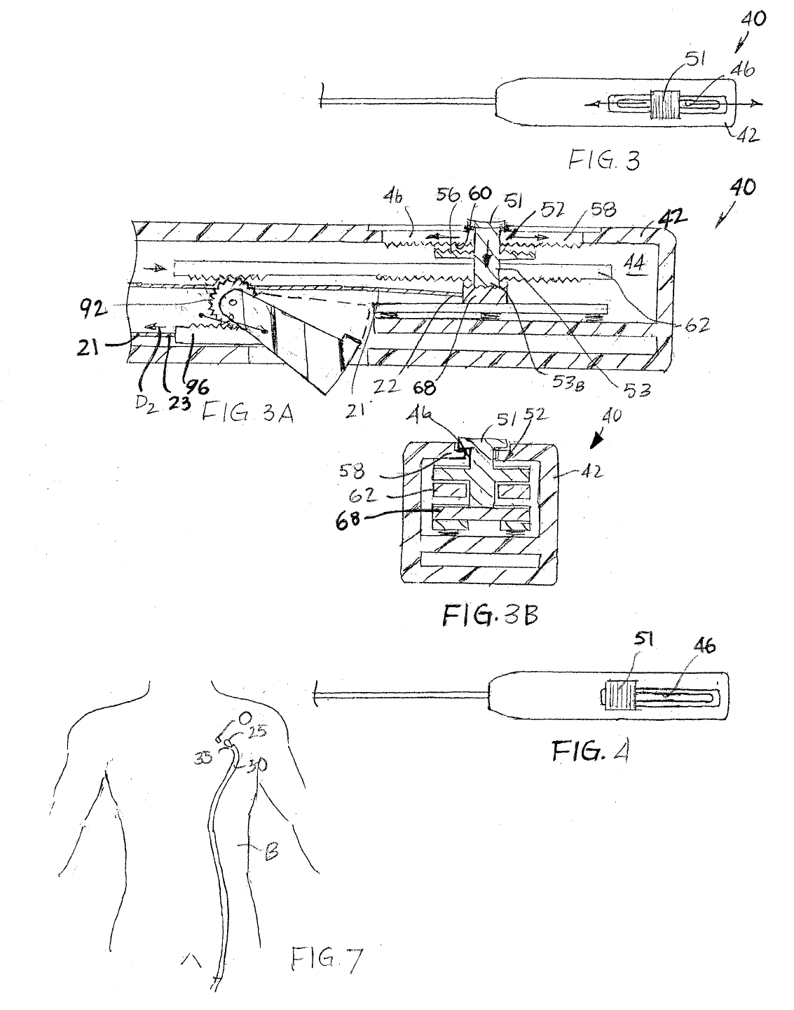

Turning now to FIG. 3, pressing the button 51 may enable it and its associated actuator 52 to travel to a selected location along a length of the elongated slot 46 in the body 42 of the handle 40. As illustrated by FIGS. 3A and 3B, when the button 51 is pressed, the actuator 52 is forced further into the interior 44 of the handle (e.g., downward, in the orientation depicted by FIGS. 3A and 3B), causing the teeth 56 of the actuator 52 to disengage the teeth 60 of the button positioning retainer 58 and, thereby, enabling movement of the button 51 and the actuator 52 along the elongated slot 46. In addition, as the button 51 is pressed, the end 53B of the shaft 53 of the actuator 52 is forced into the shuttle 68, which moves the shuttle 68 away from the runner 62 and causes the teeth 70 of the shuttle 68 to disengage teeth 66 of the runner 62. Such movement of the shuttle 68 away from the runner 62, along with engagement of the teeth 70 of the shuttle 68 by the teeth 57 that protrude from the end 53B of the shaft of 53 of the actuator 52, enables movement of the shuttle 68 along the length of the runner 62 as the button 51 and the actuator 52 are moved along the elongated slot 46. As the position of the shuttle 68 changes, so does the position of the first end 22 of the elongated element 21 of the snare 20 (FIG. 1A), which may result in a change in the size of the loop 25 (FIG. 1A) that may extend, or protrude, from the distal end 35 (FIGS. 1 and 1A) of the sheath 30 (FIGS. 1 and 1A).

When the button 51, actuator 52, and shuttle 68 have been moved to locations that provide the loop 25 (FIGS. 1 and 1A) with a desired size, the button 51 may be released, fixing its new position along the elongated slot 46, as shown in FIG. 4, and fixing the positions of the actuator 52, the shuttle 66, and the first end 22 of the elongated element 21 of the snare 20, as illustrated by the arrangement of the components of the loop size control 50 shown in FIGS. 2A and 2B.

With reference returned to FIG. 1, and to FIG. 2A, a reciprocating control 80 of the handle 40 may include a trigger 81 may cause the elongated element 21 of the snare 20 to move back and forth, or to reciprocate or oscillate, along its length. In the specific, but nonlimiting embodiment depicted by FIGS. 1 and 2A, the trigger 81 may include external element 82 located outside of the body 42 of the handle 40, an intermediate section 84 that extends form a location outside of the body 42 to a location within the interior 44 of the handle 40, and a hinge 86 adjacent to the intermediate section 84, on an opposite side of the intermediate section 84 from the external element 82. The hinge 86 may pivot about a pin 88 that extends across least a portion of the interior 44 of the handle 40. A spring 90 may be associated with the intermediate section 84 of the trigger 81 and an interior location of the body 42 of the handle 40 in a manner that biases, or forces, the trigger 81 toward an initial position P_{I}. As the trigger 81 is move from the initial position P_{I} to a depressed position P_{D}, or a pivoted position, tension may be introduced into the spring 90. When the external element 82 of the trigger 81 is released, the spring 90 releases the tension and returns the trigger 81 from the depressed position P_{D} to the initial position P_{I}.

The pin 88 may also extend through the center of one or more gears 92. Each gear 92 may be positioned adjacent to the hinge 86 (e.g., a single gear 92 may be positioned adjacent to one side of the hinge 86, a pair of gears 92 may be positioned on opposite sides of the hinge 86, etc.).

In some embodiments, teeth 94 of each gear 92 may engage corresponding, complementarily configured teeth 98 of an element 97 of a second runner 96. The second end 23 of the elongated element 21 of the snare 20 (FIGS. 1 and 1A) may be secured to the second runner 96. The second runner 96 may be located on an opposite side of the gear 92 from runner 62 (i.e., a first runner) and aligned with the first runner 62. Like the first runner 62, the second runner 96 may be capable of back and forth movement along its length, while a distance the second runner 96 is spaced apart from the first runner 62 remains constant, or fixed. Movement of the second runner 96 along its length may be enabled in any suitable manner, including, without limitation, by way of elongated recesses (not shown) in a portion of the body 42 of the handle 40 that receive opposite edges (not shown) of the second runner 96. As the trigger 81 moves between its initial position P_{I} and its depressed position P_{D} and each gear 92 associated with the trigger 81 rotates, teeth 94 of each gear 92, which engage teeth 98 of the second runner 96, may cause the second runner 96 to move in a direction that corresponds to a direction in which the gear 92 rotates. For example, as the external element 82 of the trigger 81 is pressed, it pivots from its initial position P_{I} to its depressed position P_{D}, and each gear 92 may rotate partially. As each gear 92 rotates, it may cause the second runner 96 and the second end 23 of the elongated element 21 of the snare 20 to move backwards, or proximally. Thus, pressing the external element 82 of the trigger 81 may induce movement of the snare 20 in a first direction D₁.

Teeth 94 of each gear 92 may also engage corresponding, complementary teeth 66 of a corresponding element 64 of the runner 62. Thus, as the external element 82 of the trigger 81 is released, the spring 90 associated with intermediate element 84 of the trigger 81 may cause it to pivot back to its initial position P_{I}, rotating the gear 92, which may cause the runner 62 to move backwards, or proximally, pulling the shuttle 68 and the first end 22 of the elongated element 21 of the snare 20 proximally with it. Thus, releasing the external element 82 of the trigger 81 may induce movement of the snare 20 in a second direction D₂ (FIG. 3A), opposite from the first direction D₁.

Repeatedly pressing and releasing the external element 82 of the trigger 81 may induce back and forth movement in the elongated element 21 of the snare 20, which may enable advancement of the snare 20 within the body of a subject, positioning of the snare over an object with the subject's body, engagement of an object with the body of the subject, and/or cutting of an object within the body of the subject.

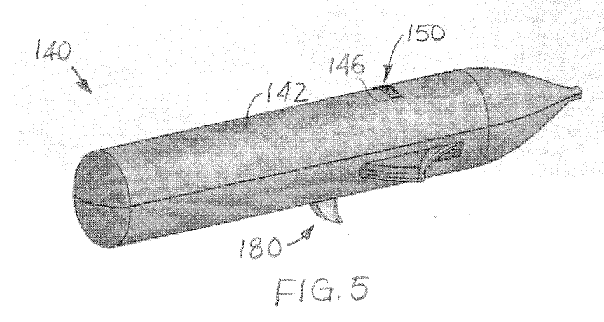

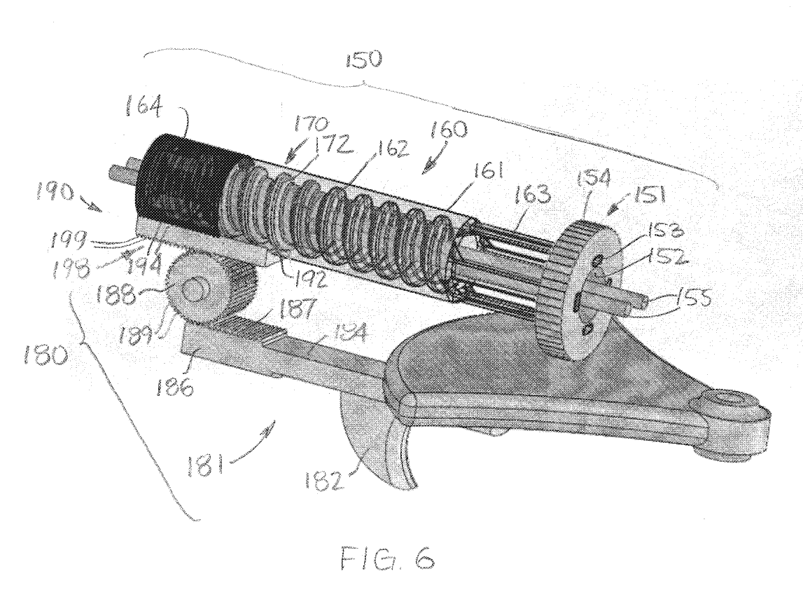

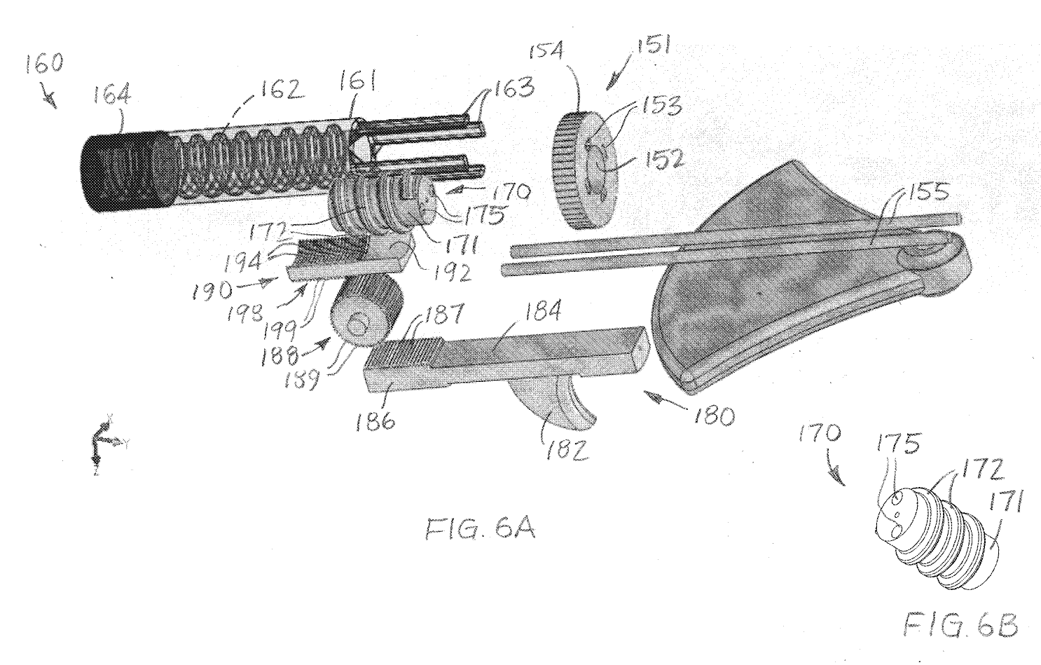

FIGS. 5, 6, 6A, and 6B illustrate another embodiment of a handle 140 that may be used with a retrieval apparatus 10 (FIG. 1) according to this disclosure. That embodiment of handle 140 includes a body 142 that carries a loop size control 150 and a reciprocating control 180.

As shown in FIG. 5, the loop size control 150 of the handle 140 includes a wheel 151 that enables an individual to adjust the size of a loop 25 of a snare 20 (FIG. 1A). A portion of the wheel 151 protrudes through a slot 146 in a body 142 of the handle 140. The location of the wheel 151 may enable an individual to engage and rotate it while with his or her thumb or a finger while the individual holds the body 142 of the handle 140 with the remainder of his or her hand. As depicted by FIGS. 6 and 6A, the wheel 151 may be associated with a barrel 160 of the loop size control 150, which barrel 160 may be generally tubular in shape. More specifically, retention apertures 153 that extend through the wheel 151 at various locations that are radially equidistant from an axis of rotation, or center, of the wheel 151 receive corresponding circumferentially arranged shafts 163 that protrude from a distal portion of the barrel 160. With this arrangement, when the wheel 151 is rotated about its center, the barrel 160 also rotates about a longitudinal axis through its center. The wheel 151 may slide longitudinally along the shafts 163 to enable the wheel 151 to remain in place along a length of the body 142 (FIG. 5) of the handle 140 (FIG. 5) when the barrel 160 is moved longitudinally within the body 142, as will be described more fully hereinafter.

As the wheel 151 and the barrel 160 rotate, their longitudinal positions along the length of the handle 140 (FIG. 5) may remain stationary or substantially stationary. An interior surface of a body 161 of the barrel 160 may include a helical groove 162. With continued reference to FIGS. 6 and 6A, and as illustrated by FIG. 6B, the helical groove 162 may receive a helical ridge 172 on an outer surface 171 of a shuttle 170 that resides within an interior of the barrel 160. The shuttle 170 of the loop size control 150 of the handle 140 may include one or more rotation prevention channels 175. Each rotation prevention channel 175 may extend through a length of the shuttle 170 and receive a corresponding elongated rotation prevention element 155. At least one end of each elongated rotation prevention element 155 may be secured to a fixed location within the body 142 (FIG. 5) of the handle 140, and each elongated rotation prevention element 155 may extend through a central aperture 152 in the wheel 151, an interior of the barrel 160, and its corresponding rotation prevention channel 175 of the shuttle 170. Each rotation prevention channel 175 may receive a corresponding elongated rotation prevention element 155 in a manner that enables the shuttle 170 to slide freely along at least a portion of the length of the elongated rotation prevention element 155. The elongated retention prevention element(s) 155 and the rotation prevention channel(s) 175 work in concert to prevent the shuttle 170 from rotating when the wheel 151 and the barrel 160 rotate. The use of a plurality of elongated retention prevention elements 155 and a corresponding plurality of rotation prevention channels 175 may prevent the shuttle 170 from rotating when the wheel 151 and the barrel 160 rotate. Alternatively, or in addition, cross-sectional shapes (e.g., non-circular cross-sectional shapes) taken along a length of at least one elongated retention prevention element 155 and along a length of its corresponding rotation prevention channel 175 may prevent the shuttle 170 from rotating while the wheel 151 and the barrel 160 rotate.

A shuttle drive element 172, such as the helical ridge of the embodiment of shuttle 170 depicted by FIGS. 6, 6A, and 6B, may function in conjunction with a complementary barrel drive element 162, such as the helical recess formed in the interior surface of the body 161 of the barrel 160, as depicted by FIGS. 6 and 6A. As the wheel 151 and the barrel 160 rotate, the elongated rotation prevention element(s) 155 and the rotation prevention channel(s) 175 through the shuttle 170 may prevent rotation of the shuttle 170, while the barrel drive element 162 and the shuttle drive element 172 may force the shuttle 170 longitudinally forward (i.e., distally) or backward (i.e., proximally) through a length of the interior of the barrel 160, depending up on the direction in which the wheel 151 and the barrel 160 are rotated.

An interface element 190 may provide for an interface between the loop size control 150 and the reciprocating control 180. The interface element 190 may be capable of enabling the reciprocating control 180 to cause reciprocation of the elongated element 21 (FIG. 1) of the snare 20 (FIG. 1), including the portion of the elongated element 21 that forms the loop 25 (FIG. 1) without adjusting the loop size control 50 and, thus, without altering the size of the loop 25.

The illustrated embodiment of the interface element 190 includes a barrel engagement surface 192 that is capable of interfacing with the barrel 160 of the loop size control 150 or that is configured to interface with the loop size control 150. More specifically, the barrel engagement surface 192 of the interface element 190 may include an elongated concave shape, such as a semi-cylindrical shape, that extends along the length of the interface element 190 and that complements a curvature of the barrel 160. A series of parallel threads 194 may protrude from the barrel engagement surface 192, with gaps or spaces between the parallel threads 194 being capable of or configured to receive a complementary series of parallel threads 164 that protrude from an outer surface of and extend around the circumference of a portion of the barrel 160. In the illustrated embodiment, the threads 164 of the barrel 160 are located around a distal portion of the barrel 160 and the barrel engagement surface 192 of the interface element 190 is, therefore, positioned adjacent to and receives the distal portion of the barrel 160 in such a way that the threads 194 of the barrel engagement surface 192 mesh with, interleave with, or mutually engage the threads 164 on the barrel 160. Of course, the threads 164 that extend around the exterior of the barrel 160 may be positioned along any other suitable portion of the barrel 160 and the barrel engagement surface 192 may be positioned against and received by that portion of the barrel 160 so that the threads 194 of the interface element 190 and the threads 164 on the barrel 160 may mesh with, interleave with, or mutually engage each other. Since the threads 164 on the barrel 160 are oriented parallel to one another and the threads 194 that protrude from the barrel engagement surface 192 of the interface element 190 are oriented parallel to one another, the threads 164 on the barrel 160 and the threads 194 of the interface element 190 may enable the barrel 160 to rotate relative to the interface element 190 while maintaining a longitudinal position of the barrel 160 relative to the interface element 190.

In addition to the barrel engagement surface 192, the illustrated embodiment of interface element 90 may include a reciprocating control interface surface 198. As depicted, the reciprocating control interface surface 98 may be opposite from (e.g., face in an opposite direction from, etc.) the barrel engagement surface 192. The reciprocating control interface surface 198 may be shaped complementarily to a shape of a corresponding feature of the reciprocating control 180, such as the gear 188 depicted by FIGS. 6 and 6A. Without limitation, the reciprocating control interface surface 198 may have a generally flat configuration. A series of parallel teeth 199 may protrude from the reciprocating control interface surface 198 of the interface element 190. The teeth 199 may be arranged at a pitch that corresponds to a pitch of the teeth 189 on an outer edge of the gear 188, with the teeth 189 of the gear 188 meshing with, interleaving with, or mutually engaging the teeth 199 of the reciprocating control interface surface 198. Thus, as the gear 188 rotates about a fixed axis, the teeth 189 of the gear 188 mesh with, interleave with, or mutually engage the teeth 199 of the reciprocating control interface surface 198, which will cause the interface element 190 to move in a direction that corresponds to the direction in which the gear 188 rotates (e.g., clockwise rotation of the gear 188 may cause the interface element 190 to move forward, or distally; counterclockwise rotation of the gear 188 may cause the interface element 190 to move backward, or proximally). Such movement of the interface element 190 may be transmitted through the threads 194 on its barrel engagement surface 192 to the threads 164 on the exterior of the barrel 160, thereby causing the barrel 160 to move in the same direction. As indicated previously herein, as the barrel 160 moves longitudinally, each of its shafts 163 may slide through its corresponding retention aperture 153 in the wheel 151, enabling the wheel 151 to remain in the same longitudinal position along the handle 140.

Movement of the gear 188 may be caused, or effected, in any suitable manner. As a non-limiting example, and as illustrated, a trigger 181 of the reciprocating control 180 may cause the gear 188 to rotate. In a more specific embodiment, the trigger 181 may be capable of longitudinal movement relative to the body 142 (FIG. 5) of the handle 140 (FIG. 5). An external element 82 of the trigger 181 may protrude from the body 142 of the handle 140 to enable an individual to engage and move the trigger 181 with his or her finger as he or she holds the body 142 of the handle 140 in a remainder of his or her hand. In the depicted embodiment, the external element 182 of the trigger 181 is capable of longitudinal movement along a length of the body 142 of the handle 140. The external element 182 of the trigger 181 is secured to or integral with an interior section 184 of the trigger 181, with the interior section 184 residing within the body 142 of the handle 140. The trigger 181 may be engaged by, and its movement confined (e.g., to two-dimensions, such as rearward and forward sliding) by corresponding features of the handle 140. In a specific embodiment, a portion of the external element 182 of the handle 181 may reside within the elongated slot 146 (FIG. 5) in the body 142 of the handle 140, with portions of a wall of the body 142 on opposite sides of the elongated slot 146, through which the elongated slot 146 is formed, being trapped between enlarged features of the external element 182 and the interior section 184 of the trigger 181 adjacent to opposite surfaces (e.g., an enlarged feature of the interior section 184 may be positioned adjacent to an interior surface of the body 142 of the handle 140; an enlarged feature of the external element 182 may be positioned adjacent to a surface that located on an outside of the body 142; etc.) of that portion of the wall of the body 142. With such an arrangement, as the external element 182 of the trigger 181 is pulled in a first direction (e.g., backward, or proximally) along the elongated slot 146 in the body 142 of the handle 140, the interior section 184 of the trigger 181 may move in the same direction. Movement of the interior section 184 and the external element 182 of the trigger 180 in an opposite, second direction (e.g., forward, or distally) may occur after the individual releases the external element 182 of the trigger 181, under the resilient force of a spring that was deformed upon forcing the external element 182 of the trigger 181 in the first direction.

The interior section 184 of the trigger 181 may include a gear engagement element 186 with teeth 187 that are complementary to and cooperate with (e.g., mesh with, interleave with, mutually engage, etc.) the teeth 189 that protrude from the edge of the gear 188. Thus, movement of the trigger 181 may cause the gear 188 to rotate in a corresponding direction (e.g., clockwise when the external element 182 of the trigger 181 is moved backward, or proximally; counterclockwise when the external element 182 of the trigger 181 moves forward, or distally), inducing corresponding movement in the interface element 190 and the barrel 160 (e.g., forward, or distally; backward, or proximally; respectively).

When the handle 140 is used as part of a retrieval apparatus 10 (see, e.g., FIG. 1A) according to this disclosure, the first end 22 (FIG. 1A) of the elongated element 21 (FIG. 1A) of the snare 20 (FIG. 1A) of the retrieval apparatus 10 may be coupled to the shuttle 170, while the second end 23 (FIG. 1A) of the elongated element 21 of the snare 20 may be coupled to the interior section 184 of the trigger 181. With such an arrangement, when the external element 182 of the trigger 181 is forced in its first direction (e.g., backwards, or proximally), the interior section 184 of the trigger 181 and the second end 23 of the elongated element 21 of the snare 20 are also pulled in the first direction, while the trigger 181 moves the barrel 160 in the opposite, second direction (e.g., forwards, or distally) by an equivalent distance, enabling the first end 22 of the elongated element 21 of the snare 20 to move the same distance in the second direction. The opposite effect occurs as the external element 182 of the trigger 181 moves in the second direction, back to its original position. The result of using the reciprocating control 180 in this manner, including when the external element 182 of the trigger 181 is repeatedly pressed and released, may be back-and-forth movement of the intermediate element 21 of the snare 20 without altering the size of the loop 25 (FIG. 1A) that protrudes from the distal end 35 (FIG. 1A) of the sheath 30 (FIG. 1A) of the retrieval apparatus 10.

Now referring to FIG. 7 an embodiment of a method of retrieving an object O from a location within a body B of a subject is illustrated. In such a method, a distal end 35 of a sheath 30 of a retrieval apparatus 10 is introduced into the body B and advanced to a location of an object O. With the distal end 35 of the sheath 30 at the location of the object O, a loop 25 of a snare 20 (FIG. 1A) of the retrieval apparatus 10, which may be sized in the manner described in reference to FIGS. 2-4, in the manner described in reference to FIGS. 5-6B, or in any other suitable manner before and/or after introduction of the sheath 30 into the body B, may be moved into place over the object O. In some embodiments, the elongated element 21 (FIG. 1A) that defines the loop 25 may be reciprocated, such as in the manner described in reference to FIGS. 1A, 2B, and 5-6B. Once the loop 25 (and, optionally, the distal end 35 of the sheath 30 by decreasing the size of the loop 25 or, in embodiments where the loop 25 may be retracted into a lumen 34 (FIG. 1A) of the sheath 30, by retracting the loop 25) has (have) engaged the object O, the sheath 30 may be pulled proximally out of the body B, pulling the loop 25 and the object O with it.

Although the foregoing description provides many specifics, these should not be construed as limiting the scope of any of the appended claims, but merely as providing information pertinent to some specific embodiments that may fall within the scopes of the appended claims. Features from different embodiments may be employed in combination. Other embodiments may also be devised which lie within the scopes of the appended claims. The scope of each claim is indicated and limited only by the appended claims and their legal equivalents. All additions, deletions and modifications to the disclosed subject matter that fall within the meaning and scopes of the claims are to be embraced by the claims.

What is claimed:

1. An apparatus for retrieving an object located within a body of a subject, comprising:
a snare comprising an elongated element with:
a first end;
a first region adjacent to the first end;
a central region adjacent to the first region, opposite from the first end;
a second region adjacent to the central region, opposite from the first region; and
a second end adjacent to the second region;
a sheath including:
a wall defining and longitudinally surrounding a lumen, the lumen extending through a length of the wall, portions of the first region and the second region of the elongated element of the snare being located within the lumen and surrounded by the wall;
a distal end from which the central region of the snare protrudes, the distal end and the central region of the snare collectively defining a loop; and
a proximal end opposite from the distal end;
a handle, including:
a housing to which the proximal end of the catheter is secured and into which the first end and the second end of the elongated element of the snare extend;
a snare sizing control associated with the first end of the elongated element of the snare;
a snare reciprocating control associated with the first end and/or the first portion of the elongated element of the snare and with the second end and/or the second portion of the elongated element of the snare;
at least one actuator carried by the housing and operably associated with the snare sizing control and the snare reciprocating control.

2. The apparatus of claim 1, wherein the snare sizing control is capable of adjusting a length of the first portion of the elongated element of the snare that resides within the housing without adjusting a length of the second portion of the elongated element of the snare that resides within the housing.

3. The apparatus of claim 2, wherein the housing includes an elongated slot and the at least one actuator comprises a button capable of being depressed and, while being depressed, sliding at least partially along a length of the elongated slot to determine a position of the first end of the elongated element of the snare relative to the length of the elongated slot.

4. The apparatus of claim 3, wherein the button is capable of locking into place along the length of the elongated slot at a position that corresponds to a position of the first end of the elongated element of the snare within the housing of the handle.

5. A method for retrieving an object from within a body of a subject, comprising:
introducing a sheath and a snare into a body of a subject to an approximate location of an object to removed from the body;
positioning a loop extending from a distal end of the sheath around the object; and
reciprocating the snare to cause the loop to cut into the object;
engaging the object; and
pulling the sheath, the snare, and the object out of the body of the subject.

6. The method of claim 5, further comprising:
engaging the object between the loop of the snare and the distal end of the sheath.

7. The method of claim 6, wherein engaging the object between the loop of the snare and the distal end of the sheath comprises at least partially retracting the loop.

8. The method of claim 6, wherein engaging the object between the loop of the snare and the distal end of the sheath comprises reducing a size of the loop.

9. The method of claim 5, further comprising:
sizing the loop.

10. The method of claim 9, wherein sizing the loop comprises sizing the loop before introducing the sheath and the snare into the body of the subject.

11. The method of claim 9, wherein sizing the loop comprises sizing the loop with the loop at the approximate location of the object.

12. The method of claim 5, further comprising:
retracting the loop into a lumen of the sheath prior to introducing the sheath and the snare into the body of the subject; and
extending the loop beyond the distal end of the sheath after introducing a distal end of the sheath to the approximate location of the object.

13. A method for retrieving an object from within a body of a subject, comprising:
adjusting a size of a loop of a snare;
introducing a sheath and the snare into a body of a subject to an approximate location of an object to removed from the body;
positioning the loop around the object; and
pulling the sheath, the snare, and the object proximally out of the body of the subject.

14. The method of claim 13, further comprising:
retracting the loop into a lumen of the sheath prior to introducing the sheath and the snare into the body of the subject; and
extending the loop beyond the distal end of the sheath after introducing a distal end of the sheath to the approximate location of the object.

15. The method of claim 14, further comprising:
engaging the object between the loop of the snare and the distal end of the sheath by at least partially retracting the loop into the lumen of the sheath once the loop has been positioned around the object.

16. The method of claim 13, further comprising:
reducing a size of the loop once the loop has been positioned around the object.

17. The method of claim 13, wherein adjusting the size of the loop comprises adjusting the size of the loop before introducing the sheath and the snare into the body of the subject.

18. The method of claim 13, wherein adjusting the size of the loop comprises adjusting the size of the loop with the loop at the approximate location of the object.

19. An apparatus for retrieving an object located within a body of a subject, comprising:
a snare comprising an elongated element with:
a first end;
a first region adjacent to the first end;
a central region adjacent to the first region, opposite from the first end;
a second region adjacent to the central region, opposite from the first region; and
a second end adjacent to the second region;
a sheath including:
a wall defining and longitudinally surrounding a lumen, the lumen extending through a length of the wall, portions of the first region and the second region of the elongated element of the snare being located within the lumen and surrounded by the wall;
a distal end from which the central region of the snare protrudes, the distal end and the central region of the snare collectively defining a loop; and
a proximal end opposite from the distal end;
a handle, including:
a housing to which the proximal end of the catheter is secured and into which the first end and the second end of the elongated element of the snare extend; and
a snare reciprocating control associated with the first end and/or the first portion of the elongated element of the snare and with the second end and/or the second portion of the elongated element of the snare.

20. The apparatus of claim 19, wherein the snare reciprocating control includes a trigger that pivots relative to the housing of the handle.
